(19) United States
(12) Patent Application Publication
(10) Pub. No.: US 2005/0281789 A1
(43) Pub. Date: Dec. 22, 2005

(54) Transcutaneous and/or transdermal transport of materials

(76) Inventors: Mangala Rao, Silver Spring, MD (US); Carl R. Alving, Bethesda, MD (US); Kristina K. Peachman, Bowie, MD (US); Stephen W. Rothwell, Columbia, MD (US)

Correspondence Address: BLANK ROME LLP, 600 NEW HAMPSHIRE AVENUE, N.W., WASHINGTON, DC, 20037, US

(21) Appl. No.: 11/133,436

(22) Filed: May 20, 2005

Related U.S. Application Data
(60) Provisional application No. 60/572,700, filed on May 20, 2004.

(51) Int. Cl. A61K 45/00; A61K 39/00

(52) U.S. Cl. 424/93.7; 424/277.1

(57) ABSTRACT

The invention relates to transcutaneous and/or transdermal transport of materials, such as antigens, drugs, drugs, nucleic acids, (e.g., DNA and RNA), proteins, other therapeutic agents, dyes, and the like, into the skin and/or the body. The material is transported transcutaneously and/or transdermally using an antigen presenting cell (APC) by applying the APC and the material on to the surface of the skin.

This application claims the benefit of provisional U.S. Provisional Patent Application No. 60/572,700, filed May 20, 2004, which is incorporated herein by reference.

### FIELD OF THE INVENTION

The invention relates to transcutaneous and/or transdermal transport of materials, such as antigens, drugs, nucleic acids, (e.g., DNA and RNA), proteins, other therapeutic agents, dyes, and the like, into the skin and/or the body.

### BACKGROUND OF THE INVENTION

Skin, the largest human organ, plays an important part in the body's defense against invasion by infectious agents and contact with noxious substances. But this barrier function of the skin appears to have prevented the art from appreciating that transcutaneous immunization provided an effective alternative to enteral, mucosal, and parenteral administration of vaccines.

Anatomically, skin is composed of three layers: the epidermis, the dermis, and subcutaneous fat. Epidermis is composed of the basal, the spinous, the granular, and the cornified layers; the stratum corneum comprises the cornified layer and lipid. The principal antigen presenting cells of the skin, Langerhans cells, are reported to be in the mid- to upper-spinous layers of the epidermis in humans. Dermis contains primarily connective tissue. Blood and lymphatic vessels are confined to the dermis and subcutaneous fat.

The stratum corneum (SC), a layer of dead skin cells and lipids, has traditionally been viewed as a barrier to the hostile world, excluding organisms and noxious substances from the viable cells below the SC. The SC also serves as a barrier to the loss of moisture from the skin: the relatively dry SC is reported to have 5% to 15% water content while deeper epidermal and dermal layers are relatively well hydrated with 85% to 90% water content. The basis of the barrier properties of the outermost layer of skin lies in the unique characteristics of the SC. The SC is thought to have a “bricks and mortar” structure, in which the bricks are viewed as overlapping layers of dead corneocyte cells derived from the epidermis, and the mortar is viewed as lipid bilayers that fill the spaces between the cells. Because the lipid bilayers, comprised mainly of a variety of ceramides and cholesterol, constitute only a small volume when compared with the corneocytes, the continuity of passage of this element of the SC is not only highly hydrophobic, but also tortuous, convoluted, and narrow throughout the SC, and these properties constitute further factors that provide both waterproofing and substantial barrier properties for prevention of penetration of water-soluble molecules and drugs. Although the existence of a pore-pathway through the SC for hydrophilic molecules has long been debated, delivery of such large molecules is generally achieved only through transient channels opened by disruptive physical strategies, such as iontophoresis, ultrasound, photomechanical stress waves, etc.

Only recently has the secondary protection provided by antigen presenting cells (e.g., Langerhans cells) been recognized. Moreover, the ability to immunize through the skin with or without penetration enhancement (i.e., transcutaneous immunization) using a skin-active adjuvant has only been recently described. Although undesirable skin reactions such as atopy and dermatitis were known in the art, recognition of the therapeutic advantages of transcutaneous immunization (TCI) might not have been appreciated in the past because the skin was believed to provide a barrier to the passage of molecules larger than about 500 Daltons.

Transcutaneous immunization (TCI) has been proposed as a means in the art to immunize an individual by placing an immunizing agent on the outer surface of the skin (Glenn et al. Skin immunization made possible by cholera toxin. Nature 391(6670):851 (1998); Glenn et al. Transcutaneous immunization: A human vaccine delivery strategy using a patch. Nature Medicine 2000:6(12): 1403-1406 (2000); and Glenn et al. Transcutaneous immunization and immunostimulant strategies: capitalizing on the immunocompetence of the skin. Expert Reviews of Vaccines 2(2):253-267, 2003). Patents disclosing TCI includes WO 98/20734, WO 99/43350, WO 00/61184; U.S. Pat. Nos. 5,910,306 and 5,980,898; and U.S. Patent Application Publication Nos. 2004/0028727, 2004/0146534, 2004/0258703, 2004/0047872, 2004/0137004, and 2004/0185055; which are incorporated herein by reference. TCI typically has been accomplished either by hydration of the skin by using an occlusive patch to moisten the SC, or by mild abrasion of the upper layer of the SC (Glenn et al., 2003). The process of TCI does allow relatively large molecules (e.g., proteins) to penetrate into the skin to a sufficient level to induce an immune response, but there is still an upper limit on the size of substance that can penetrate. Even at the level of the size of a killed virus, such as with an influenza virus vaccine, penetration is limited or nonexistent, as judged by the immune response (Glenn et al., 2003). In addition, the ability to delivery DNA by TCI is severely limited and inconsistent, presumably due to poorly characterized barrier properties of the SC. The cause of the poor performance by DNA for TCI is unknown, but it could be due to the strong anionic charge on the DNA molecules, the large size of the DNA molecules or molecular aggregates, or degradation of the DNA by enzymes or other factors during passage of the DNA through the skin. Regardless of the possibility of delivery of proteins or nucleic acids through the skin by using the process of hydration, there are no known channels or pores that are normally able to permit the penetration of a particle as large as a cell. In fact, one of the most characteristic properties of SC is that it serves as a strong barrier to prevent invasion and internal infection caused by virtually all forms of microorganism.

There remains a need for reliable delivery of large molecules, drugs, vaccines and other immunizing materials, including DNA, through the skin. There is further a need to protect the delivered materials from degradation or adverse changes during their passage through the skin. One way to accomplish these needs would be to enclose the material that would passage through the skin inside a cell that would transit into and through the skin from the outside-in. This procedure would serve to protect the material to be delivered from deleterious effects from enzymes or other factors that might adversely affect the delivery, and it would allow the delivery of large substances, such as virus-size particles or DNA. Moreover, the delivery cell would not degrade or adversely affect the desired biological or medical activities of the delivered substance.

### SUMMARY OF THE PRESENT INVENTION

Novel and inventive formulations for transcutaneous and/or transdermal delivery of materials, especially antigens, as well as processes for making and using them, are disclosed herein. The stratum corneum (SC) has been proposed to be a composite material with a high flexibility that can behave as a biopolymer or a membrane. Because of its ability to maintain its structure and function in the face of environmental stresses or damage, the SC is disclosed herein as a “smart” system, that is both passively smart (with properties of selectivity, shapeability, self-recovery, simplicity, self-repair, and stability) and actively smart (senses ambient changes, uses feedback system, makes a useful response). The concept of a smart system characterized by considerable adaptability also applies to many internal systems in the body, most notably, the immune system. The cellular elements of the immune system have unique characteristics that allow them to undergo cooperative interactions in mounting an immune response against an externally introduced threat or foreign material such as a bacterial infection, or to an internal threat such as cancer. One of the characteristics of certain cells in the immune system is that they may have mobility and have the ability to traverse across tissues or spaces that are not available to other cells.

Certain cells also may have a natural affinity for a particular tissue or location, but also may have flexibility in that upon stimulation they may have the capacity to conduct signals from an outside source to stimulate or amplify inherent useful biological systems in the body. An example of such a cell is the Langerhans cell (LC), a cell that resides in the epidermis below the SC, and that constitutes a vital element of the skin immune system. It expresses high levels of major histocompatibility complex (MHC) class II molecules and has a strong stimulatory function for the activation of T lymphocytes. The LC has the ability to differentiate into a dendritic cell, and it serves as a potent antigen-presenting cell for initiation of an immune response. Dendritic cells are characterized by dendritic appearance after culture. Although they resemble macrophages, they have a lesser, but not absent, ability to ingest materials by phagocytosis. All of the various dendritic cell and macrophage types share the antigen presentation and phagocytosis abilities, and could represent cell types that could serve as carriers of drugs and vaccines across the skin barriers. All of these cells are also characterized by mobility, and this property would allow delivery of materials to draining lymph nodes and local circulation that would lead to delivery of the cells and their contents to distant locations in the body.

Because of the above properties, antigen presenting cells (APC), such as LCs, dendritic cells, and macrophages, which have properties that allow them to respond to varying conditions in the environment and to transduce signals for stimulation of other systems, most particularly the immune system, could be referred used as “smart” cells to be used as carriers of materials across the SC from outside-in for the purpose of inducing an immune response, or for the purpose of delivering drugs or nucleotides to distant internal locations for preventive or therapeutic purposes. Because of the natural location of these cells in the epithelium below the SC, there may be an unknown driving force, either a chemical or physical affinity, that directs a migratory tendency of such a cell placed on the outer surface of the skin toward the epithelial space beneath the SC through unknown pores or mechanisms that would allow such a transitional movement from the outside-in. For example, LCs, dendritic cells, and macrophages are excellent cells for the delivery of materials into the lymphatic system and other systems that receive lymphatic circulation.

An embodiment of the present invention is to provide transcutaneous and/or transdermal delivery of materials, such as large molecules (>500 Daltons), drugs, therapeutic agents, vaccines, and the like, by using an APC as a carrier of the large molecules into and/or across the skin barrier. This system provides simple application of a formulation having an APC cell and the material to the surface of the skin. Preferably, the APC cell is incubated with the material, so that the material is incorporated into or become associated with the APC cell, prior to application to the skin. Once applied to the skin, the APC serves as a carrier to transport the materials transcutaneously and/or transdermally.

In particular, the present invention provides for transcutaneous and/or transdermal immunization (TCI) by using an APC as a carrier of antigen across the skin barrier. This system provides simple application of a composition having an APC cell and the antigen to the surface of the skin. Preferably, the APC cell is incubated with the antigen, so that the antigen is incorporated into the APC cell, prior to application to the skin.

### DETAILED DESCRIPTION OF THE PREFERRED EMBODIMENT

The present invention uses antigen presenting cells (APC) as carriers to deliver materials, such as large molecules (>500 Daltons), drugs, therapeutic agents, and vaccines, through the skin. In a preferred embodiment, the APC is incubated with the material prior to its application to the surface on intact skin. The incubation allows the APC to uptake and to incorporate the material. Incubation of the APC and the material preferably takes place in a buffer, most preferably phosphate buffered saline (PBS), at an appropriate temperature and pH for both the APC and the material, most preferably about 30-40° C. and pH=6.5-8.0. The incubation is timed so that the APC has sufficiently up take the material to be transported. The cell can then be used to apply to the skin of an animal for transcutaneous delivery of the material. In a preferred embodiment, the APC is first concentrated and resuspended in a solution prior to application to the skin.

The present invention can be practiced with or without skin penetration. For example, chemical or physical penetration enhancement techniques may be used as long as the skin is not perforated through the dermal layer. Hydration of the intact or skin before, during, or immediately after application of the formulation is preferred and may be required in some or many instances. For example, hydration may increase the water content of the topmost layer of skin (e.g., stratum corneum or superficial epidermis layer exposed by penetration enhancement techniques) above 25%, 50% or 75%.

Skin may be swabbed with an applicator (e.g., adsorbent material on a pad or stick) containing hydration or chemical penetration agents or they may be applied directly to skin. For example, aqueous solutions (e.g., water, saline, other buffers), acetone, alcohols (e.g., isopropyl alcohol), detergents (e.g., sodium dodecyl sulfate), depilatory or keratinolytic agents (e.g., calcium hydroxide, salicylic acid, ureas), humectants (e.g., glycerol, other glycols), polymers (e.g., polyethylene or propylene glycol, polyvinyl pyrrolidone), or combinations thereof may be used or incorporated in the formulation. Similarly, abrading the skin (e.g., abrasives like an emery board or paper, sand paper, fibrous pad, pumice), removing a superficial layer of skin (e.g., peeling or stripping with an adhesive tape), microporating the skin using an energy source (e.g., heat, light, sound, electrical, magnetic) or a barrier disruption device (e.g., gun, microneedle), or combinations thereof may act as a physical penetration enhancer. See WO98/29134, which is incorporated herein by reference, for microporation of skin; and U.S. Pat. No. 6,090,790, which is incorporated herein by reference, for microneedles; and U.S. Pat. No. 6,168,587, which is incorporated herein by reference, for transdermal guns which might be adapted for use in transcutaneous vaccination. The objective of chemical or physical penetration enhancement in conjunction with present invention is to remove at least the outer most epidermal layer without perforating the skin through to the dermal layer. This can be accomplished with minor discomfort at most to the human or animal subject and without bleeding at the site. For example, applying the formulation to intact skin may not involve thermal, optical, sonic, or electromagnetic energy to perforate layers of the skin below the SC or epidermis.

The materials to be transported can be, but is not limited to, large molecules (>500 Daltons), drugs, therapeutic agents, and vaccines. For vaccines, antigens can be derived from any pathogen that infects a human or animal subject (e.g., bacterium, virus, fungus, or protozoan). The chemical structure of the antigen may be described as one or more of carbohydrate, fatty acid, and protein (e.g., glycolipid, glycoprotein, lipoprotein). Proteinaceous antigen is preferred. The molecular weight of the antigen may be greater than 500 Daltons, 800 Daltons, 1000 Daltons, 10 kiloDaltons, 100 kiloDaltons, or 1000 kiloDaltons. Chemical or physical penetration enhancement may be preferred for macromolecular structures like cells, viral particles, and molecules of greater than one megaDalton (e.g., CS6 antigen), but techniques like hydration and swabbing with a solvent may be sufficient to deliver the material across the skin. Antigen may be obtained by recombinant techniques, chemical synthesis, or at least partial purification from a natural source. It may be a chemical or recombinant conjugates, for example, linkage between chemically reactive groups or protein fusion. Antigen may be provided as a live cell or virus, an attenuated live cell or virus, a killed cell, or an inactivated virus. Alternatively, antigen may be at least partially purified in cell-free form (e.g., cell or viral lysate, membrane or other subcellular fraction). Because most adjuvants would also have immunogenic activity and would be considered antigens, adjuvants would also be expected to have the aforementioned properties and characteristics of antigens.

The vaccine can also include genetic materials, i.e. nucleic acids, such as DNA or RNA. Genetic immunization has been described in U.S. Pat. Nos. 5,589,466, 5,593,972, and 5,703,055, which are incorporated herein by reference. The nucleic acid(s) contained in the formulation may encode the antigen, the adjuvant, or both. The nucleic acid may or may not be capable of replication; it may be non-integrating and non-infectious. For example, the nucleic acid can encode a fusion polypeptide comprising antigen and a ubiquitin domain to direct the immune response to a class I restricted response. The nucleic acid can further comprise a regulatory region operably linked to the sequence encoding the antigen or adjuvant. The nucleic acid can be added with an adjuvant. The nucleic acid can be complexed with an agent that promotes transfection such as cationic lipid, calcium phosphate, DEAE-dextran, polybrene-DMSO, or a combination thereof. Immune cells can be targeted by conjugation of DNA to Fc receptor or protein A/G, or attaching DNA to an agent linking it to α₂-macroglobulin or protein A/G or similar APC targeting material.

The formulation can also contains an adjuvant, although a single molecule may contain both adjuvant and antigen properties (e.g., E. coli heat-labile enterotoxin). Adjuvants are substances that are used to specifically or non-specifically potentiate an antigen-specific immune response, perhaps through activation of antigen presenting cells. Although activation may initially occur in the epidermis or dermis, the effects may persist as the dendritic cells migrate through the lymph system and the circulation. Adjuvant may be formulated and applied with or without antigen, but generally, activation of antigen presenting cells by adjuvant occurs prior to presentation of antigen. Alternatively, they may be separately presented within a short interval of time but targeting the same anatomical region (e.g., the same draining lymph node field). The adjuvant can be added during the incubation process of the APC and the antigen or just before application of the formulation to the skin. Alternatively, the adjuvant and the formulation can be applied to the same region of the skin separately.

Adjuvants include, for example, chemokines (e.g., defensins, HCC-1, HCC4, MCP-1, MCP-3, MCP4, MIP-1α, MIP-1β, MIP-1δ, MIP-3α, MIP-2, RANTES); other ligands of chemokine receptors (e.g., CCR1, CCR-2, CCR-5, CCR-6, CXCR-1); cytokines (e.g., IL-1.beta., IL-2, IL-6, IL-8, IL-10, IL-12; IFN-γ; TNF-α; GM-CSF); other ligands of receptors for those cytokines, immunostimulatory CpG motifs in bacterial DNA or oligonucleotides; muramyl dipeptide (MDP) and derivatives thereof (e.g., murabutide, threonyl-MDP, muramyl tripeptide); heat shock proteins and derivatives thereof; Leishmania homologs of eIF4a and derivatives thereof; bacterial ADP-ribosylating exotoxins and derivatives thereof (e.g., genetic mutants, A and/or B subunit-containing fragments, chemically toxoided versions); chemical conjugates or genetic recombinants containing bacterial ADP-ribosylating exotoxins or derivatives thereof; C3d tandem array; lipid A and derivatives thereof (e.g., monophosphoryl or diphosphoryl lipid A, lipid A analogs, AGP, AS02, AS04, DC-Chol, Detox, OM-174); ISCOMS and saponins (e.g., Quil A, QS-21); squalene; superantigens; or salts (e.g., aluminum hydroxide or phosphate, calcium phosphate). See also Nohria et al., Biotherapy 7:261-269, 1994, and Richards et al., in Vaccine Design, Eds. Powell et al., Plenum Press, 1995, for other useful adjuvants.

The immune response induced by the present invention may include the elicitation of antigen-specific antibodies and/or lymphocytes. Antibody can be detected by immunoassay techniques. Detection of the various antibody isotypes (e.g., IgM, IgD, IgA1, IgA2, secretory IgA, IgE, IgG1, IgG2, IgG3, or IgG4) can be indicative of a systemic or regional immune response. Immune responses can also be detected by a neutralizing assay. Antibodies are protective proteins produced by B lymphocytes. They are highly specific, generally targeting one epitope of an antigen. Often, antibodies play a role in protection against disease by specifically reacting with antigens derived from the pathogens causing the disease. Immunization may induce antibodies specific for the immunizing antigen (e.g., bacterial toxin).

CTL are immune cells produced to protect against infection by a pathogen. They are also highly specific. Immunization may induce CTL specific for the antigen, such as a synthetic oligopeptide based on a malaria protein, in association with self-major histocompatibility antigen. CTL induced by immunization with the transcutaneous delivery system may kill pathogen-infected cells. Immunization may also produce a memory response as indicated by boosting responses in antibodies and CTL, lymphocyte proliferation by culture of lymphocytes stimulated with the antigen, and delayed type hypersensitivity responses to intradermal skin challenge of the antigen alone.

Successful protection could also be demonstrated by challenge studies using infection by the pathogen or administration of toxin, or measurement of a clinical criterion (e.g., high antibody titers or production of IgA antibody-secreting cells in mucosal membranes may be used as a surrogate marker).

Besides vaccines, the present invention can also be used to deliver other materials. Preferably those material cannot, by themselves, penetrate the skin by topical application, which are generally larger than about 500 Daltons, and include, but are not limited to, drugs (e.g. anticancer agents and antibiotics), prodrugs, nucleic acids, (e.g., DNA and RNA), proteins, other therapeutic agents, dyes, radioactive substances, and the like. These materials are transported transcutaneously and/or transdermally for therapeutic or diagnostic purposes other than to elicit an immune response.

According to the present invention, the materials to be carried by the APC can be enclosed in a liposome prior to incubation with the APC. Liposomal systems for delivery of vaccines or drug are known in the art and are disclosed, for example, in U.S. Pat. No. 5,910,306, which is incorporated herein by reference. Overall, liposomes are generally closed vesicles surrounding an internal aqueous compartment. The internal compartment carries the materials to be delivered and is separated from the external medium by a lipid bilayer composed of discrete lipid molecules. They can be composed of a variety of lipid components such as, for example, phospholipid, nonionic surfactant, synthetic or natural lipid, saturated or unsaturated lipid, and charged or neutral lipid, either with or without a sterol. Liposomes may be either multilamellar, paucilamellar, or unilamellar, and may be made in different sizes: small being less than 25 nm, intermediate being 25 nm to 500 nm, and large being greater than 500 nm. A typical liposome is composed of dimyristoyl phosphatidylcholine (DMPC), dimyristoyl phosphatidylglycerol (DMPG), and cholesterol, with or without lipid A, in a multilamellar configuration, and has a population of sizes from about 0.2 μm to about 10 μm. Preferably, once the material is encapsulated in the liposome, it is then incubated with the APC prior application to the skin for transcutaneous and/or transdermal delivery into the skin and/or the body.

Without further description, it is believed that one of ordinary skill in the art can, using the preceding description and the following illustrative examples, make and utilize the compounds of the present invention and practice the claimed methods. The following example is given to illustrate the present invention. It should be understood that the invention is not to be limited to the specific conditions or details described in this example.

### EXAMPLE 1

### Preparation of The Formulation

The following procedure is carried out under sterile conditions in a biological safety cabinet. Non-adherent purified murine dendritic cells are placed in sterile 6 mL polypropylene tubes. Dendritic cells (1×10⁶ to 6×10⁶ cells) are incubated with vaccine antigens (approximately 50 μg/mL) in a total volume of 1 mL of sterile phosphate buffered saline (PBS), pH=7.2 for at least 90 min at 37° C. in a CO₂ incubator. After 90 min, 3 mL of sterile PBS is added to the dendritic cells. Cells are spun by centrifugation at 1200 rpm in a refrigerated bench top centrifuge for 10 min. The supernatant is discarded and the cell pellet gently dislodged by tapping and 4 mL of sterile PBS is added to re-suspend the cells. Cells are centrifuged as described above. The cell pellet is re-suspended in a small volume of PBS. Cells are now ready for application on the skin.

### EXAMPLE 2

### Transdermal Transport of Fluorescent Dye Using Dendritic Cells

Dendritic cells were obtained by culturing the marrow from the femur and tibia of BALB/c mice using published protocols. Dendritic cells (2.9×10⁶ cells) were labeled with PKH26 red fluorescent dye for 5 min at RT and then washed thoroughly in RPMI-1640 complete media followed by PBS.

A BALB/c mouse was anaesthetized with Ketamine and Rompamine. The right ear of the mouse was flattened out by adhering it to a petri dish using a piece of double-sided scotch tape. The dorsal surface of the ear was rubbed 4 times with sand paper (used for EKG) followed by hydration. 30 μl of sterile water was applied onto the ear surface and a saturated cotton swab was used to spread the water across the ear, but not all the way to the edges. The water was allowed to sit for 5 minutes and then blotted dry with a dry swab. After prepping the ears, the antigen (30 μl) was added with a pipet tip. The labeled dendritic cells (2.9×10⁶) were applied to the ear and allowed to remain on the ear for 1 hour. The remaining solution containing the dendritic cells was removed with dry sterile cotton swabs. The ear was swabbed thoroughly with a cotton swab saturated with sterile water. Excess water was removed with a dry cotton swab and the mouse was placed on a heating pad and allowed to revive. The left ear served as the negative control. After 24 h, the mouse was euthanized and the ears, spleen and lymph node were obtained for experimental analysis. Cryo sections were obtained from portions of the ears and spleen and examined using a fluorescence microscope. Single cell suspensions were also made from the spleen and lymph nodes and examined by flow cytometry and fluorescence microscopy. Fluorescent cells were observed in the spleen (6.97%,), lymph nodes (0.43%) and the treated ear. No fluorescent cells were observed in the untreated left ear (control).

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

1. A method for transdermal and/or transcutaneous transport of a material comprising the step of applying a composition to a skin surface of an organism, wherein the composition comprises an antigen presenting cell (APC) and the material.

2. The method of claim 1, wherein the APC is incubated with the material prior to the applying step.

3. The method of claim 1, wherein the APC is selected from the group consisting of Langerhans cell, dendritic cell, macrophage.

4. The method of claim 1, wherein the material has a molecular weight greater than 500 Daltons.

5. The method of claim 1, wherein the material is selected from the group consisting of drugs, prodrugs, therapeutic agents, and antigens.

6. The method of claim 5, wherein the antigen is derived from a pathogen, a tumor cell, a normal cell, or a pathogen.

7. The method of claim 5, wherein the antigen is a tumor antigen.

8. The method of claim 5, wherein the antigen is an autoantigen.

9. The method of claim 5, wherein the antigen is selected from the group consisting of carbohydrate, glycolipid, glycoprotein, lipid, lipoprotein, peptide, phospholipid, and protein.

10. The method of claim 5, wherein the antigen is obtained by recombinant means, purification, or chemical synthesis.

11. The method of claim 5, wherein the antigen is a peptide or a protein.

12. The method of claim 1, wherein the composition further comprises an adjuvant.

13. The method of claim 1, wherein the material is incorporated into the APC.

14. The method of claim 13, wherein the material is encapsulated in a liposome.

15. A method for making a formulation for transcutaneous and/or transdermal delivery comprising the steps of incubating an antigen presenting cell (APC) with a material to be delivered.

16. The method of claim 15, wherein the APC is selected from the group consisting of Langerhans cell, dendritic cell, macrophage.

17. The method of claim 15, wherein the material has a molecular weight greater than 500 Daltons.

18. The method of claim 15, wherein the material is selected from the group consisting of therapeutic agents, drugs, prodrugs, DNA, and antigens.

19. The method of claim 18, wherein the antigen is derived from a pathogen, a tumor cell, a normal cell, or a pathogen.

20. The method of claim 18, wherein the antigen is a tumor antigen.

21. The method of claim 18, wherein the antigen is an autoantigen.

22. The method of claim 18, wherein the antigen is selected from the group consisting of carbohydrate, glycolipid, glycoprotein, lipid, lipoprotein, peptide, phospholipid, and protein.

23. The method of claim 18, wherein the antigen is obtained by recombinant means, purification, or chemical synthesis.

24. The method of claim 18, wherein the antigen is a peptide or a protein.

25. The method of claim 15, wherein the composition further comprises an adjuvant.

26. The method of claim 15, wherein the material is incorporated into the APC.

27. The method of claim 15, wherein the material is encapsulated in a liposome.

28. A composition for transcutaneous and/or transdermal transport of a material comprising an antigen presenting cell (APC) and the material.

29. The composition of claim 28, wherein the APC is selected from the group consisting of Langerhans cell, dendritic cell, macrophage.

30. The composition of claim 28, wherein the material has a molecular weight greater than 500 Daltons.

31. The composition of claim 28, wherein the material is selected from the group consisting of therapeutic agents, drugs, prodrugs, DNA, and antigens.

32. The composition of claim 31, wherein the antigen is derived from a pathogen, a tumor cell, a normal cell, or a pathogen.

33. The composition of claim 31, wherein the antigen is a tumor antigen.

34. The composition of claim 31, wherein the antigen is an autoantigen.

35. The composition of claim 31, wherein the antigen is selected from the group consisting of carbohydrate, glycolipid, glycoprotein, lipid, lipoprotein, peptide, phospholipid, and protein.

36. The composition of claim 31, wherein the antigen is obtained by recombinant means, purification, or chemical synthesis.

37. The composition of claim 31, wherein the antigen is a peptide or a protein.

38. The composition of claim 28, wherein the composition further comprises an adjuvant.

39. The composition of claim 28, wherein the material is incorporated into the APC.

40. The composition of claim 28, wherein the material is encapsulated in a liposome.
